(19) United States
(12) Patent Application Publication
(10) Pub. No.: US 2025/0369963 A1
(43) Pub. Date: Dec. 4, 2025

(54) DETECTION OF TARGET ANALYTE IN SAMPLE

(71) Applicant: D12 SCIENTIFIC LLC, Danville, KY (US)

(72) Inventor: Daniel F. Scott, Danville, KY (US)

(21) Appl. No.: 19/106,278

(22) PCT Filed: Sep. 1, 2023
(86) PCT No.: PCT/US2023/073313

§ 371 Date: Feb. 25, 2025

Related U.S. Application Data
(60) Provisional application No. 63/403,361, filed on Sep. 2, 2022.

(51) Int. Cl. G01N 33/543; C12Q 1/6823

(52) U.S. Cl. CPC G01N 33/54326; C12Q 1/6823; G01N 33/54346

(57) ABSTRACT

A device, method, and kit are disclosed for use in detecting a target analyte in a sample. The device includes a region having a first zone, into which the sample can be delivered, and a second zone in fluid communication with the first zone. A magnet is positioned adjacent the first zone, and a magnetic nanoparticle is held within the first zone by the magnet. The magnetic nanoparticle includes a magnetic core. The device further makes use of a recognition element that is covalently or non-covalently conjugated to a signal component, and a probe that is covalently or non-covalently conjugated to the recognition element, or taken together with the recognition element, forms a single unit. The probe, the recognition element, or the signal component has an affinity for the target analyte, such that in the presence of the target analyte, the signal component is free from or does not attach to the magnetic core, allowing the signal component to move away from the first zone.

### RELATED APPLICATIONS

This application claims priority from U.S. Provisional Application Ser. No. 63/403,361 filed Sep. 2, 2022, the entire disclosure of which is incorporated herein by this reference.

### GOVERNMENT INTEREST

This invention was made with government support under grant number 8P20GM103436 awarded by the National Institutes of Health. The government has certain rights in the invention.

### TECHNICAL FIELD

The presently-disclosed subject matter generally relates to on-site detection of a target analyte in a sample. In particular, certain embodiments of the presently-disclosed subject matter relate on-site sample analysis tools, which are adaptable and user-friendly, and which can be used in any convenient location, including the site of a sample of interest, or in a location where a subject providing a biological sample is located.

### INTRODUCTION

Providing on-site sample analysis can address a number of important needs. On-site detection tools allow for environmental testing of water, soil, or air samples in their native location without the need to collect and transport samples to a laboratory. In the context of patient care, on-site detection tools such as point-of-care (POC) diagnostic systems provide the opportunity for testing at a convenient location, and shorten the time from analysis to diagnosis, thereby improving care and treatment decisions.

As will be appreciated, in some circumstances, the ability to provide for POC diagnostic systems can be critical for patient care. For example, deployed military personal or individuals who are operating in remote locations are often unable to physically relocate or to deliver uncompromised samples to a facility having diagnostic laboratory equipment. Additionally, traditionally-marginalized communities, rural communities, and socioeconomically-challenged communities carry a disproportionate burden from both communicable and noncommunicable diseases, which is due in part to the lack of early diagnosis and pathology services, which are critical for early detection, diagnosis, and disease management. Traditionally-marginalized communities may also be less likely to visit and trust medical professionals. Greater access to effective POC diagnostic systems could help individuals in these and other circumstances, providing access to quick, portable, and simple diagnostic and analytical feedback. Opportunities for telehealth would also greatly benefit from the ability to take a diagnostic test at home and upload or report the results to a medical professional.

Increased availability of POC diagnostic devices for use at home or in remote areas, without a need for trained professionals, could encourage individuals to seek medical care while providing information for initial diagnosis and disease management. As exemplified by the COVID-19 pandemic, rapid analysis can greatly slow the spread of communicable disease by identifying infected individuals.

Early examples of POC systems include a 1950s-era dipstick formulated for glucose quantification. Since then, POC devices have evolved to become common in everyday life, including glucose monitors for diabetics and at-home pregnancy tests. Current POC systems offer the advantages of portability and quick readout compared to traditional laboratory-based analysis but still suffer from several issues, including complicated protocols that may be difficult for untrained personnel to complete, limited stability and shelf life of biological components, the requirement of specialized instrumentation, susceptibility to matrix effects, and limited ability to detect certain analytes.

Accordingly, there remains a need in the art for improved on-site sample analysis tools, which are adaptable and user-friendly, and which can be used in any convenient location, including the site of a sample of interest (such as the site of testing water, soil, air quality, etc.) or in a location where a patient is located (such as when a biological sample will be tested). For diagnostic and other applications involving a biological sample from a patient, such a device would be particularly valuable to those who are unable or less likely to seek medical care in a traditional office setting, and for use in remote regions where advanced medical instrumentation and lab analysis are not available.

### SUMMARY

The presently-disclosed subject matter meets some or all of the above-identified needs, as will become evident to those of ordinary skill in the art after a study of information provided in this document.

The presently-disclosed subject matter includes a device for detecting a target analyte in a sample. The device includes a region having a first zone, into which the sample can be delivered, and a second zone in fluid communication with the first zone. A magnet is positioned adjacent the first zone, and a magnetic nanoparticle is held within the first zone by the magnet. The magnetic nanoparticle includes a magnetic core. The device further makes use of a recognition element that is covalently or non-covalently conjugated to a signal component, and a probe that (i) is covalently or non-covalently conjugated to the recognition element, or (ii) taken together with the recognition element, forms a single unit. The probe, the recognition element, or the signal component has an affinity for the target analyte, such that in the presence of the target analyte, the signal component is free from or does not attach to the magnetic core, allowing the signal component to move away from the first zone.

A device of the presently-disclosed subject matter includes (a) a region having a first zone, into which the sample can be delivered, and a second zone in fluid communication with the first zone; (b) a magnet positioned adjacent the first zone; and (c) a magnetic nanoparticle having a magnetic core, held within the first zone by the magnet.

In some embodiments, the device also includes a signal component, a recognition element that is covalently or non-covalently conjugated to the signal component, and a probe that (i) is covalently or non-covalently conjugated to the recognition element, or (ii) taken together with the recognition element, forms a single unit. In such embodiments of the device, the probe, the recognition element, or the signal component has an affinity for the target analyte, or the target analyte cleaves the recognition element. In this regard, in the presence of the target analyte, the signal component is free from the magnetic core, such that it can migrate away from the magnetic core and the first zone of the device.

In some embodiments of the device, the probe is covalently or non-covalently conjugated to the magnetic core.

In some embodiments of the device, in which the probe is conjugated to the magnetic core, the recognition element has an affinity for the probe, and a stronger affinity for the target analyte, such that (i) in the absence of the target analyte, the signal component is bound to the magnetic core due to the affinity between the probe and the recognition element, and (ii) in the presence of the target analyte, the signal component is free from the magnetic core due to the stronger affinity between the recognition element and the target analyte. For example, in some embodiments, the target analyte can be a target nucleotide, the probe can be a probe nucleotide, and the recognition element can be a recognition nucleotide conjugated to the probe nucleotide through complementary base-pairing, wherein the recognition nucleotide has an affinity for the probe nucleotide, and a stronger affinity for the target nucleotide. For another example, in some embodiments, the target analyte can be recognized by an aptamer, the probe is a probe nucleotide, and the recognition element is a nucleotide aptamer conjugated to the probe nucleotide through complementary base-pairing, wherein the nucleotide aptamer has an affinity for the probe nucleotide, and a stronger affinity for the target analyte.

In some embodiments of the device, in which the probe is conjugated to the magnetic core, the probe has an affinity for the recognition element, and a stronger affinity for the target analyte, such that (i) in the absence of the target analyte, the signal component is bound to the magnetic core due to the affinity between the probe and the recognition element, and (ii) in the presence of the target analyte, the signal component is free from the magnetic core due to the stronger affinity between the probe and the target analyte. For example, in some embodiments, the target analyte can be a target nucleotide, the probe can be a probe nucleotide; and the recognition element can be a recognition nucleotide conjugated to the probe nucleotide through complementary base-pairing, wherein the probe nucleotide has an affinity for the recognition nucleotide, and a stronger affinity for the target nucleotide.

In some embodiments of the device, in which the probe is conjugated to the magnetic core, the target analyte is a protease, the probe and the recognition element, taken together, form a single unit that is a polypeptide, and the recognition element is an amino acid sequence recognized by the protease for cleaving, such that, (i) in the absence of the target protease, the signal component is bound to the magnetic core due to polypeptide remaining intact, and (ii) in the presence of the target protease, polypeptide is cleaved, such that the signal component is free from the magnetic core.

In some embodiments of the device, in which the probe is conjugated to the magnetic core, the target analyte is a polypeptide or small molecule, the recognition element has an affinity for the signal component, and a stronger affinity for the target analyte, such that, (i) in the absence of the target analyte, the signal component is bound to the magnetic core due to the affinity between the recognition element and the signal component; and (ii) in the presence of the target polypeptide, the signal component is free from the magnetic core due to the stronger affinity between the recognition element and the target polypeptide.

In some embodiments of the device, in which the probe is conjugated to the magnetic core, the target analyte is a polypeptide or small molecule, the signal component has an affinity for the recognition element, and a stronger affinity for the target analyte, such that, (i) in the absence of the target analyte, the signal component is bound to the magnetic core due to the affinity between the recognition element and the signal component; and (ii) in the presence of the target polypeptide, the signal component is free from the magnetic core due to the stronger affinity between the signal component and the target polypeptide.

In some embodiments of the device, in which the probe is conjugated to the magnetic core, the target analyte can be recognized by an aptamer, and the recognition element is an aptamer, and the probe and the recognition element, taken together, form a single unit non-covalently attached to the magnetic core, wherein the aptamer has a stronger affinity for the target analyte than for the magnetic core, such that, (i) in the absence of the target analyte, the signal component is bound to the magnetic core due to the affinity between the aptamer and the magnetic core, and (ii) in the presence of the target analyte, the signal component is free from the magnetic core due to the stronger affinity between the aptamer and the target analyte.

In some embodiments, the device also includes target analytes covalently or non-covalently bound to the magnetic core, and a prepared sample. The prepared sample can include a recognition element and a probe, taken together to form a single unit attached to the signal component, such that (i) in the absence of the target analyte in the sample, the signal component is bound to the magnetic core due to the affinity between the single unit and the target analytes bound to the magnetic core, and (ii) in the presence of the target analyte in the sample, the signal component is free to migrate away from the magnetic core because the single unit was bound to the target analyte in the sample, such that it is unable to bind the target analytes bound to the magnetic core.

In some embodiments of the device in which target analytes are bound to the magnetic core, the target analyte can be recognized by an antibody or fragment thereof, and the single unit includes an antibody or fragment thereof that selectively binds the target analyte. In other embodiments, the target analyte can be recognized by an aptamer, and the single unit includes an aptamer that selectively binds the target analyte. In other embodiments, the target analyte can be recognized by a nucleotide, and the single unit includes a nucleotide that selectively binds the target analyte. In other embodiments, the target analyte can be recognized by a polypeptide, and the single unit includes a polypeptide that selectively binds the target analyte.

The presently-disclosed subject matter further includes a method for detecting a target analyte in a sample, which involves delivering the sample to a device as disclosed herein such that the sample enters the first zone, and detecting a location of the signal component, such that (i) in the absence of the target analyte, the signal component is bound to the magnetic core and held within the first zone, and (ii) in the presence of the target analyte, the signal component is free from the magnetic core, allowing movement away from the first zone.

The presently-disclosed subject matter further includes a kit for detecting a target analyte in a sample, which includes (a) a device having a region, comprising (i) a first zone, into which the sample can be delivered, and a second zone in fluid communication with the first zone; (i) a magnet positioned adjacent the first zone; (iii) a magnetic nanoparticle having a magnetic core, held within the first zone by the magnet; (b) a probe; and (c) a recognition element that can be covalently or non-covalently conjugated to a signal component. Some embodiments of the kit can include a signal component. In other embodiments, the signal component can be separately obtained and used with the kit.

In some embodiments of the kit, a device is provided in which the probe is covalently or non-covalently conjugated to the magnetic core. In some embodiments, the probe is also provided together with the recognition element to form a single unit.

In some embodiments of the kit, the probe and/or recognition element are provided separately from the device, such as in a separate container. In some embodiments, the probe and the recognition element, taken together, form a single unit. In some embodiments, the single unit is provided in a container for contacting with the sample prior to being introduced to the device. In some embodiments, the kit also includes a signal component. In some embodiments, the signal component is conjugated to the single unit.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A and 1B are schematics illustrating the operation of an exemplary embodiment of the presently-disclosed subject matter in the absence of a target analyte (FIG. 1A) and in the presence of a target analyte (FIG. 1B).

FIG. 2 is a schematic illustrating an embodiment of a magnetic nanoparticle provided in accordance with the presently-disclosed subject matter for use in detecting a target analyte that is a nucleotide.

FIG. 3 is a schematic illustrating an embodiment of a magnetic nanoparticle provided in accordance with the presently-disclosed subject matter for use in detecting a target analyte that is a polypeptide having proteolytic enzymatic activity.

FIG. 4 is a schematic illustrating an embodiment of a magnetic nanoparticle provided in accordance with the presently-disclosed subject matter for use in detecting a target analyte that is a polypeptide.

FIG. 5 is a schematic illustrating another embodiment of a magnetic nanoparticle provided in accordance with the presently-disclosed subject matter for use in detecting a target analyte that is a polypeptide.

FIG. 6 is a schematic illustrating an embodiment of a magnetic nanoparticle provided in accordance with the presently-disclosed subject matter for use in detecting a target analyte that is a small molecule, polypeptide, or other analyte that can be recognized by an aptamer.

FIG. 7 is a schematic illustrating another embodiment of a magnetic nanoparticle provided in accordance with the presently-disclosed subject matter for use in detecting a target analyte that is a small molecule, polypeptide, or other analyte that can be recognized by an aptamer.

FIG. 8 is a schematic illustrating an embodiment of a magnetic nanoparticle provided in accordance with the presently-disclosed subject matter for use in detecting a target analyte that is a small molecule, polypeptide, or other analyte that can be recognized by an antibody or fragment thereof.

FIG. 9A-9D are related to studies illustrating utility of an embodiment directed to detection of an exemplary target nucleotide.

FIG. 10 is related to studies illustrating utility of an embodiment directed to detection of an exemplary target nucleotide.

FIG. 11A-11C are related to studies illustrating utility of an embodiment directed to detection of an exemplary target polypeptide that is a protease.

FIG. 12 is related to studies illustrating utility of an embodiment directed to detection of a target analyte using an aptamer-based detection system.

FIG. 13 is related to studies illustrating utility of an embodiment directed to detection of a target analyte using an antibody-based detection system.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes devices, methods, and magnetic nanoparticles for use in detecting a small molecule, a nucleotide, or a polypeptide target analyte in a sample. Relevant samples include any that may contain a target analyte of interest. Examples of relevant samples include biological samples, such as urine, serum, blood, plasma, saliva, sputum, feces, tear, hair, nails, whole cells, soil, water, air, manufacturing materials, and food/beverage industry samples. In some embodiments, the sample could be a non-fluid sample, in which case it could be prepared prior to analysis, for example, by using appropriate reagents, such as solubilization reagents and lysis buffers. In some embodiments, sample preparation could also include extraction (e.g., whole cell extraction; extraction from aqueous or organic solutions), filtration (e.g., to remove red blood cells), and/or amplification steps (e.g., nucleotide amplification by PCR).

As will be appreciated by one of ordinary skill in the art, where biological samples are being employed, the device and method disclosed herein has diagnostic and prognostic applications. For example, embodiments of the presently-disclosed subject matter could be used in connection with alpha-1 antitrypsin, a deficiency of which can lead to severe lung and liver disease, could be detected. For another example, embodiments of the presently-disclosed subject matter could be used in connection with high-risk genotypes of HPV 16 and HPV 18/45, which are associated with 74% of cervical cancer cases. For another example, embodiments of the presently-disclosed subject matter could be used in connection with detection and risk assessment of prostate cancer. For another example, embodiments of the presently-disclosed subject matter could be used in connection with pre-diabetes screen and kidney function. For another example, embodiments of the presently-disclosed subject matter could be used in connection with identification of bacterial or yeast populations in the ethanol production industry. For another example, embodiments of the presently-disclosed subject matter could be used in connection with the detection of cortisol levels in saliva of human or other mammal subjects. Of course, there are many applications for other sample types, for example, samples obtained from water, soil, or air sources. Additional examples include, for example, samples obtained in connection with the food and beverage industry, including the distillery industry, and samples obtained in the manufacturing industry or chemical production industry.

The device of the presently-disclosed subject matter includes a region having a first zone, into which the sample can be delivered, and a second zone in fluid communication with the first zone. A magnet is positioned adjacent the first zone, and a magnetic nanoparticle is held within the first zone by the magnet. The magnetic nanoparticle includes a magnetic core, a probe conjugated to the magnetic core, a signal component, and a recognition element conjugated to each of, and connecting, the probe and the signal component. The probe, the recognition element, or the signal component of the magnetic nanoparticle has an affinity for the target analyte, such that in the presence of the target analyte, the signal component is free from or does not attached to the magnetic core, allowing the signal component to move away from the first zone.

The structure and material of the device employed in connection with the presently-disclosed subject matter can vary, so long as it includes the features as disclosed herein. Examples of types of devices that could be employed include, but are not limited to, lateral flow immunoassay (LFIA) devices, lateral flow devices including, for example, nitrocellulose membranes, capillary tube, paper-based microfluidic devices (uPAD), other microfluidic devices such as, for example, lab-on-a-chip or lab-on-a-disc devices. As will be appreciated by the skilled artisan upon studying this document, in certain embodiments of the device of the presently-disclosed subject matter, the first zone and second zone that are in fluid communication could be part of a multi-channel or multi-zone region or chamber having any of a variety of patterns for multiplexed analysis. Such analysis, and preferential direction with the multi-channel or multi-zone design could be performed with volume based, pressure based, centripetal force based and/or time based strategies. Also contemplated are multiplexed analyses involving manipulation of the signal component (e.g., changing the fluorophore attached, wherein each fluorophore would correspond to a particular target analyte).

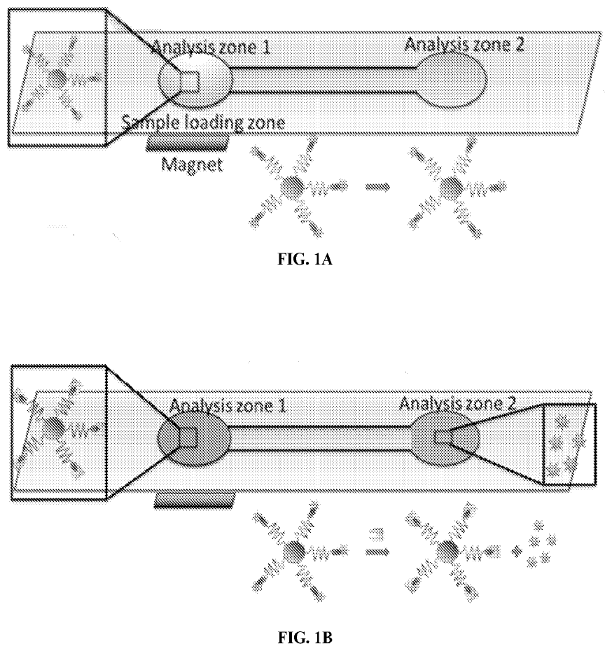

Operation of an embodiment of a device of the presently-disclosed subject matter is described with reference to FIGS. 1A and 1B. The exemplary device includes a region having a first zone, identified in FIGS. 1A and 1B as “analysis zone 1” and “sample loading zone,” into which a sample is delivered for detecting a target analyte in that sample. Also provided in this first zone is a magnetic nanoparticle, which is held within the first zone by a magnet placed adjacent the first zone. The magnet can be printed onto, or otherwise affixed to, the device and can be composed of any magnetic material. Examples of magnets that can be used include, but are not limited to neodymium magnets and rare earth magnets.

As disclosed herein, the magnetic nanoparticle includes a magnetic core, a probe conjugated to the magnetic core, a signal component, and a recognition element joining the probe and the signal component. The magnetic core can be composed of any appropriate magnetic material known in the art. Examples of magnetic particles that can be used in accordance with the presently-disclosed subject matter include, but are not limited to, polymer formulations containing magnetic components, cobalt-containing particles, nickel-containing particles, manganese-containing particles, and iron (Fe)-containing particles, such as, for example, (Fe(CO)₅, Fe[N(SiMe₃)₂]₂, Fe₃O₄, and Fe₃O₄@Au (core@shell).

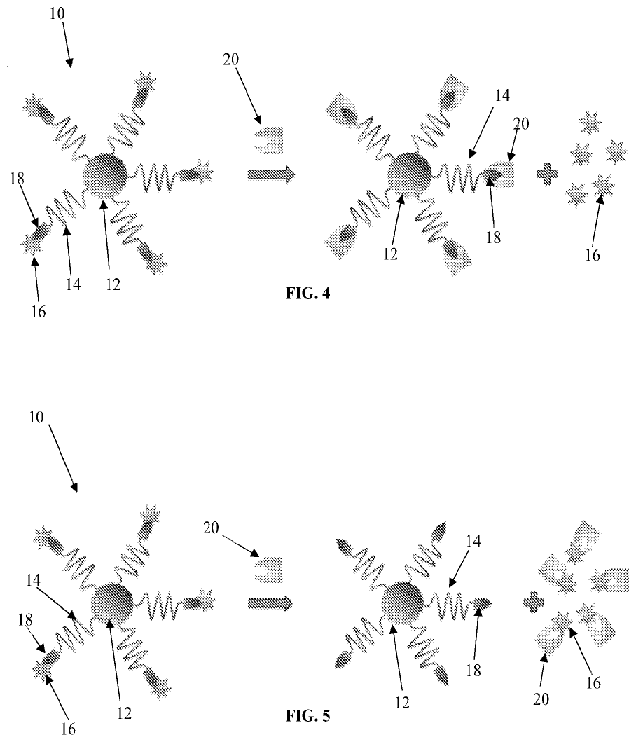

As will be appreciate by one of ordinary skill in the art upon study of this document, the probe, recognition element, and signal component can take different forms depending on the target analyte and the desired operation of the device. The exemplary magnetic nanoparticle pictured in FIGS. 1A and 1B can also be found in FIG. 4, which is described in more detail below. Notwithstanding distinct embodiments of the magnetic nanoparticle, as disclosed herein, the probe, the recognition element, or the signal component of the magnetic nanoparticle has an affinity for the target analyte, such that in the presence of the target analyte, the signal component is free from the magnetic core, allowing the signal component to move away from the first zone.

With continued reference to FIGS. 1A and 1B, the exemplary device also includes a second zone, identified in as “analysis zone 2,” which is in fluid communication with the first zone and towards which the signal component (also referred to herein as “flare”) can migrate when it is free from the magnetic core of the magnetic nanoparticle.

As will be appreciated by one of ordinary skill in the art, any number of signal components can be employed, so long as they are capable of being attached to a nucleotide or to a polypeptide and capable of producing a detectable signal. Examples of signal components that can be used in accordance with the presently-disclosed subject matter include, but are not limited to, fluorescent molecules, colorimetric, dyes, nanoparticles, magnetic molecules, electro chemical molecules, redox-active molecules, mass-based tags, and combinations thereof. With regard to fluorescent molecules, there are many examples that will be known to one of ordinary skill in the art, including polypeptide and small molecule examples. Cy3 and Cy5 fluorophores are two examples. There are also many examples of dyes that could be employed, which include, but are not limited to, acridine, anthraquinone, azo, thiazole, and phenol based dyes. Examples of nanoparticles that can be used will also be known to one of ordinary skill in the art and include, but are not limited to gold (Au), quantum dots, and cobalt (Co) nanoparticles. Magnetic molecules can also be used with examples including, but not limited to, manganese (Mn), gadolinium (Gd), iron oxide, and platinum (Pt) compounds. As will be appreciated by the skilled artisan, various devices could be employed to detect the signal component, depending on the type of signal component being used. Examples include fluorimeters, such as a charge coupled device (CCD) or a photomultiplier tube (PMT) detector with a light-emitting diode (LED) or other light source. Additional examples include nuclear magnetic resonance (NMR) spectrometer, x-ray fluorescence spectrometer, infrared (IR) spectrometer, mass spectrometer, color or light detecting cameras or sensors, and resistance/current/potential electrochemical detection.

Referring again to FIG. 1A, when the sample is added to the device, the magnetic nanoparticle will be held in the first zone (analysis zone 1/sample loading zone) by the magnet. However, upon the introduction of a sample including the target analyte, the interaction of the probe, the recognition element, or the signal component with the remainder of the nanoparticle is altered, resulting in the signal component being free from (either alone or together with other components of the nanoparticle) the magnetic core. With reference to FIG. 1B, when the signal component is free from the magnetic core, the magnetic core will continue to be held in the first zone, while the signal component can migrate away from the first zone, e.g., to a second zone (analysis zone 2). Therefore, detection of the signal component outside of the first zone is indicative of a presence and/or amount of the target analyte in the sample.

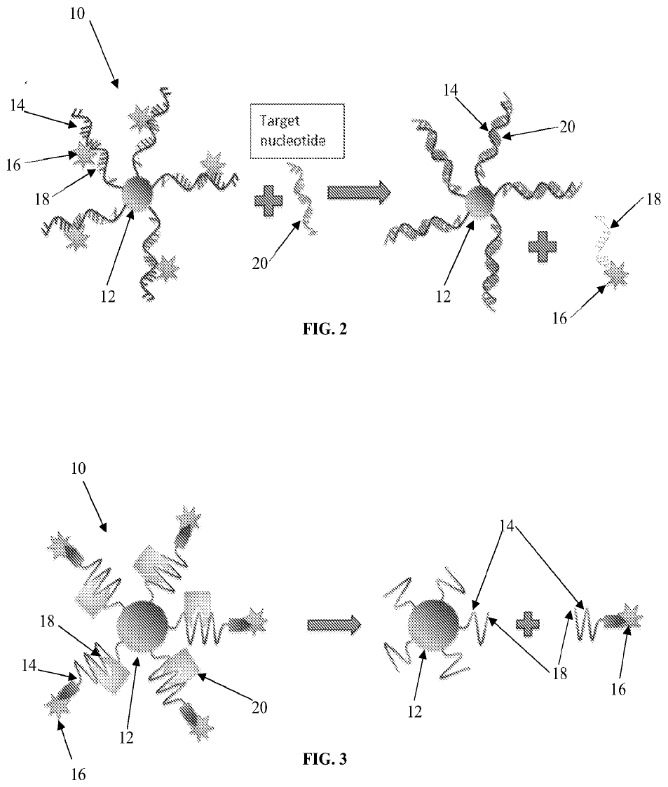

As noted above, depending on the nature of the target analyte, the probe, recognition element, and signal component can take different forms. As will be appreciated by one of ordinary skill in the art upon studying this document, there are a number of different molecule that can have utility for use as the probe and/or the recognition element of the presently disclosed subject matter. Examples include, but are not limited to nucleotides, such as DNA, for use in detecting nucleotide; polypeptides, for detecting enzymatic proteins; antibodies, for detecting proteins; binding proteins (binding polypeptides) for detecting proteins, small molecules, and nucleotide analytes; antigens, for detection of antibody- or antibody-like proteins, aptamers for detecting, small molecules, peptides, or proteins, molecularly imprinted polymers (MIP), for detecting proteins or small chemical molecule. Examples will be discussed in more detail with reference to FIG. 2-8.

Embodiments of the presently-disclosed subject matter can be used for detecting target analytes that are nucleotides or polypeptides. FIG. 2 depicts an exemplary embodiment of a magnetic nanoparticle for use in detecting a target analyte 20 that is a nucleotide. The assembled nanoparticle 10 is depicted in the left portion of FIG. 2, and includes a magnetic core 12, a probe 14, a signal component 16, and a recognition element 18.

The probe 14 is a nucleotide conjugated to the magnetic core 12. Such conjugation can be achieved by methods known to those of ordinary skill in the art, for example, by those previously described.¹⁵⁻²⁰

The recognition element 18 is a nucleotide and is conjugated to the signal component 16. Such conjugation can be achieved by methods known to those of ordinary skill in the art, for example, by those previously described in methods accessible at the following link: www.thermofisher.com/us/en/home/references/molecular-probes-the-handbook/nucleic-acid-detection-and-genomics-technology/labeling-oligonucleotides-and-nucleic-acids.html.

The recognition element 18 is conjugated to the probe 14 through complementary base-pairing. Notably, in the embodiment depicted in FIG. 2, the probe 14 has an affinity for the recognition element 18, but the probe and/or recognition element nucleotides have been selected and/or engineered such that the probe 14 has a stronger affinity for the target nucleotide 20 than for the recognition element 18. In other embodiments, the probe and/or recognition element nucleotides can be engineered such that the recognition element has a stronger affinity for the target nucleotide than for the probe. Designing nucleotides having the desired differential affinities, in view of the sequence of the target analyte, can be accomplished by modifying the length and thus number of complementary bases, nucleotide base content, and/or overall % complementary bases (to include mismatched base(s)).

With reference to the left portion of FIG. 2, when a sample containing the target nucleotide 20 is introduced, because the probe 14 has a stronger affinity for the target nucleotide 20 than for the recognition element 18, the target nucleotide 20 binds the probe 14 and displaces the recognition element 18. Through this process, the signal component 16, which is conjugated to the recognition element 18, is free from the magnetic core 12. Thus, as depicted, there is a resulting particle including the magnetic core 12, the probe 14, and the target nucleotide 20 that will remain held in the first zone by the magnet; and there is another resulting particle including the signal component 16 and the recognition element 18 that is free to migrate away from the first zone. Therefore, detection of the signal component 16 outside of the first zone will occur when the sample contains the target nucleotide 20.

As noted above, in other embodiments, the probe and/or recognition element nucleotides can be engineered such that the recognition element has a stronger affinity for the target nucleotide than for the probe. In such an embodiment, when a sample containing the target nucleotide is introduced, because the recognition element has a stronger affinity for the target nucleotide than for the probe, the target nucleotide binds the recognition element, displacing it from the probe. Through this process, the signal component, which is conjugated to the recognition element that is bound to the target nucleotide, is free from the magnetic core. Thus, there is a resulting particle including the magnetic core and the probe that will remain held in the first zone by the magnet; and there is another resulting particle including the signal component, the recognition element, and the target nucleotide that is free to migrate away from the first zone. Therefore, detection of the signal component outside of the first zone will occur when the sample contains the target nucleotide.

FIG. 3 depicts an exemplary embodiment of a magnetic nanoparticle for use in detecting a target analyte 20 that is a polypeptide that is a protease. The assembled nanoparticle 10 is depicted in the left portion of FIG. 3, and includes a magnetic core 12, a probe 14, a recognition element 18, and a signal component 16.

In the embodiment depicted in FIG. 3, the probe 14 and the recognition element 18 are provided as a single polypeptide. In this regard, the recognition element 18 portion of the polypeptide is an amino acid sequence recognized by the target protease 20 for cleaving, such that, in the presence of the target protease 20, the polypeptide is cleaved. Polypeptide sequences can be determined for specific protease recognition from the literature describing the protease discovery and/or characterization, as exemplified but not limited examples previously published.²¹⁻²³

One end of the polypeptide 14, 18 is conjugated to the magnetic core 12, while the other end of the polypeptide 14, 18 is conjugated to the signal component 16. Accordingly, and with reference to the left portion of FIG. 3, when the amino acid sequence that is contained within the polypeptide 14, 18 is recognized by the target protease 20 and cleaved there are two resulting particles. One resulting particle includes the magnetic core 12, to which a portion of the cleaved polypeptide 14, 18 is conjugated, which that will remain held in the first zone by the magnet. The other resulting particle includes the signal component 16, to which another portion of the cleaved polypeptide 14, 18 is conjugated. Therefore, detection of the signal component 16 outside of the first zone will occur when the sample contains the target protease 20.

As noted, in the example depicted in FIG. 3, one end of the polypeptide 14, 18 is conjugated to the magnetic core 12, while the other end of the polypeptide 14, 18 is conjugated to the signal component 16. Such conjugation can be achieved by methods known to those of ordinary skill in the art, for example, by those previously described to attach proteins to the surface of nanoparticles with a gold coating¹² and to fluorescently label the polypeptide signal component.¹³

FIG. 4 depicts an exemplary embodiment of a magnetic nanoparticle for use in detecting a target analyte 20 that is a polypeptide, which can be a protein that is not a protease. The assembled nanoparticle 10 is depicted in the left portion of FIG. 4, and includes a magnetic core 12, a probe 14, a recognition element 18, and a signal component 16. In the embodiment depicted in FIG. 4, the signal component 16 can be a labelled ligand binding domain of the target protein 20, which is initially attached to the probe 14 via the recognition element 18.

The conjugation between the signal component 16 and the recognition element 18 can occur through non-covalent interactions with the target polypeptide's 20 ligand, but could also be conjugated by making use of molecularly imprinted polymers, or by other mechanism. Whatever the mechanism, notably, in the embodiment depicted in FIG. 4, the recognition element 18 has an affinity for the signal component 16, but the recognition element 18 and the signal component 16 have been selected and/or engineered such that the recognition element 18 has a stronger affinity for the target polypeptide 20 than for the signal component 16. Designing a recognition element and signal component having the desired differential affinities, in view of the target polypeptide, can be accomplished by computational modelling of the protein-ligand interactions as described by Du, et al.,²⁴ or by experimental methods, including but not limited to those discussed by Zer, et al., Parker, et al., and Nguyen, et al.²⁵⁻²⁷

With reference to the left portion of FIG. 4, when a sample containing the target polypeptide 20 is introduced, because the recognition element 18 has a stronger affinity for the target polypeptide 20 than for the signal component 16, the target polypeptide 20 binds the recognition element 18 and displaces the signal component 16. Through this process, the signal component 16 is free from the magnetic core 12.

Thus, as depicted, there is a resulting particle including the magnetic core 12, the probe 14, the recognition element 18, and the target polypeptide 20 that will remain held in the first zone by the magnet. Meanwhile, the signal component 16 is free to migrate away from the first zone. Therefore, detection of the signal component 16 outside of the first zone will occur when the sample contains the target polypeptide 20.

FIG. 5 depicts another exemplary embodiment of a magnetic nanoparticle for use in detecting a target analyte 20 that is a polypeptide, which can be a protein that is not a protease. The assembled nanoparticle 10 is depicted in the right portion of FIG. 5, and includes a magnetic core 12, a probe 14, a recognition element 18, and a signal component 16. As with the embodiment depicted in FIG. 4, in the embodiment depicted in FIG. 5, the signal component 16 can be a labelled ligand of the target protein 20, which is initially attached to the probe 14 via the recognition element 18.

Notably, in the embodiment depicted in FIG. 5, the signal component 16 has an affinity for the recognition element 18, but the recognition element 18 and the signal component 16 have been selected and/or engineered such that the signal component 16 has a stronger affinity for the target polypeptide 20 than for the recognition element 18. Designing a recognition element and signal component having the desired differential affinities, in view of the target polypeptide, can be accomplished as describe with reference to the embodiment depicted in FIG. 4.

With reference to the left portion of FIG. 5, when a sample containing the target polypeptide 20 is introduced, because the signal component 16 has a stronger affinity for the target polypeptide 20 than for the recognition element 18, the target polypeptide 20 binds and displaces the signal component 16. Through this process, the signal component 16 is free from the magnetic core 12.

Thus, as depicted, there is a resulting particle including the magnetic core 12, the probe 14, and the recognition element 18 that will remain held in the first zone by the magnet. Meanwhile, the signal component 16, to which the target polypeptide 20 is bound, is free to migrate away from the first zone. Therefore, detection of the signal component 16 outside of the first zone will occur when the sample contains the target polypeptide 20.

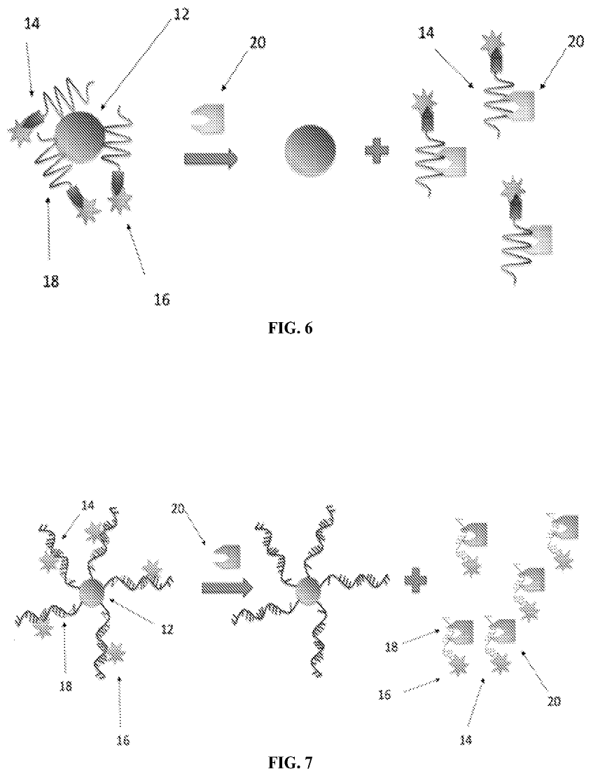

FIG. 6 depicts an exemplary embodiment of a magnetic nanoparticle for use in detecting a target analyte 20 that is a small molecule, polypeptide, or other analyte that can be recognized by an aptamer.

As is known to those of ordinary skill in the art, an aptamer is often a single-stranded nucleic acid (DNA or RNA) or sometimes a polypeptide that is designed to bind to a specific target analyte with high affinity and specificity. Aptamers are sometime referred to as chemical antibodies or synthetic receptors due to their ability to mimic the targeting capabilities of antibodies in a wide range of applications.

Aptamers can be created, for example, using a process known as Systematic Evolution of Ligands by Exponential Enrichment (SELEX). During SELEX, a large library of random nucleic acid sequences is exposed to the target analyte of interest. The sequences that bind most strongly to the target are isolated, amplified, and subjected to several rounds of selection and amplification. This iterative process eventually leads to obtaining aptamers with strong binding affinity to the target analyte.

Aptamers can be tailored to bind to a diverse array of targets, including, for example, proteins/peptides/polypeptides, nucleotides, small molecules, virus particles, whole cells, metal ions, and biological compounds and macromolecules, such as carbohydrates, lipids, lipoproteins, and other complex biomolecular assemblies. Accordingly, they can be used to detect and quantify specific analytes in complex mixtures.

In the embodiment depicted in FIG. 6, the probe 14 and the recognition element 18 are provided as a single unit. In this regard, the recognition element 18 portion is an aptamer. The assembled nanoparticle 10 is depicted in the left portion of FIG. 6, in which the aptamer-containing single unit (aptamer unit) 14, 18 is attached to the magnetic core 12, such as through electrostatic or other non-covalent interactions. In the assembled nanoparticle 10, the aptamer-containing single unit 14,18 is also attached to the signal component 16, such as by covalent conjugation.

In the embodiment depicted in FIG. 6, the aptamer unit 14,18 and signal component 16 are initially attached to the magnetic core 12, but the aptamer unit 14,18 has been selected and/or engineered such that the aptamer unit 14,18 has a stronger affinity for the target analyte 20 than for the magnetic core 12. Designing an aptamer having the desired differential affinities, in view of the target analyte, can be accomplished as previously described.^{28, 29}

With reference to the left portion of FIG. 6, when a sample containing the target analyte 20 is introduced, because the aptamer unit 14,18 has a stronger affinity for the target analyte 20 than for the magnetic core 12, the target analyte 20 binds and displaces the aptamer unit 14,18 to which the signal component 16 is conjugated. Through this process, the signal component 16 and aptamer unit 14,18 are free from the magnetic core 12.

Thus, as depicted, there is a resulting particle including the magnetic core 12 that will remain held in the first zone by the magnet. Meanwhile, the signal component 16, to which the aptamer unit 14,18 and target analyte 20 are bound, is free to migrate away from the first zone. Therefore, detection of the signal component 16 outside of the first zone will occur when the sample contains the target analyte 20.

FIG. 7 depicts an exemplary embodiment of a magnetic nanoparticle for use in detecting a target analyte 20 that is a small molecule, polypeptide, or other analyte that can be recognized by an aptamer. The assembled nanoparticle 10 is depicted in the left portion of FIG. 7, and includes a magnetic core 12, a probe 14, and a recognition element 18 conjugated to a signal component 16. In the embodiment depicted in FIG. 7, the recognition element 18 is an aptamer.

With continued reference to the embodiment in FIG. 7, the aptamer recognition element 18 is conjugated to the probe 14 through complementary base-pairing. Notably, the

The recognition element 18 has an affinity for the probe 14, but the probe and/or recognition element nucleotides have been selected and/or engineered such that the recognition element 18 has a stronger affinity for the target analyte 20 than for the probe 14.

With reference to the left portion of FIG. 7, when a sample containing the target analyte 20 is introduced, because the recognition element 18 has a stronger affinity for the target analyte 20 than for the probe 14, the target analyte 20 binds and displaces the recognition element 18 to which the signal component 16 is conjugated. Through this process, the signal component 16 and the recognition element 18 are free from the magnetic core 12.

Thus, as depicted, there is a resulting particle including the magnetic core 12 and the probe 14 that will remain held in the first zone by the magnet. Meanwhile, the signal component 16, to which the recognition element 18 and target analyte 20 are bound, is free to migrate away from the first zone. Therefore, detection of the signal component 16 outside of the first zone will occur when the sample contains the target analyte 20.

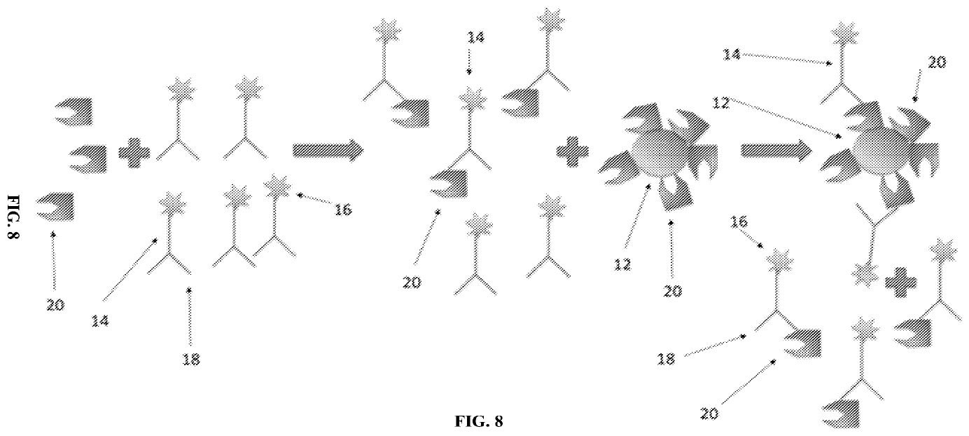

FIG. 8 depicts an exemplary embodiment of a magnetic nanoparticle for use in detecting a target analyte 20 that is a small molecule, polypeptide, or other analyte that can be recognized by an antibody, which term is inclusive of an antibody fragment (e.g., F_{ab} portion of an antibody).

In the embodiment depicted in FIG. 8, the assembled nanoparticle 10, as it exists prior to introduction of the sample to the device, is in the middle of the figure, and include a magnetic core 12 to which a target analyte 20 has been covalently or non-covalently bound.

Prior to being introduced to the device, the sample is prepared as follows. With reference to the left portion of FIG. 8, the probe 14 and the recognition element 18 are provided as a single unit. In this regard, the recognition element 18 portion is an antibody that selectively binds the target analyte 20. The antibody-containing single unit (antibody unit) 14,18 is also attached to the signal component 16, such as by covalent conjugation.

While the embodiment depicted in FIG. 8 makes use of an antibody, as will be appreciated by the skilled artisan, in other embodiments, any other probe/recognition element could be used.

To prepare the sample for introduction to the device, it is contacted with the antibody unit 14,18 attached to the signal component 16. If there is target analyte 20 in the sample, it will start to bind with the antibody 18. If there is not target analyte 20 in the sample, the antibody 18 will remain unbound.

The prepared sample is then introduced to the device containing the assembled nanoparticle 10 with the magnetic core 12 presenting the target analyte 20 (see middle of FIG. 8). If there was not target analyte 20 in the sample, the antibody unit 14,18 is free and will bind the target analyte 20 on the magnetic core 20, such that the attached signal component 16 is held in the first zone by the magnet. However, if there was target analyte 20 in the sample, it was bound to the antibody unit 14,18 during sample preparation, in which case the antibody unit 14,18 cannot become bound to the target analyte 20 on the magnetic core 12.

Thus, when the target analyte 20 is present in the sample, there is a resulting particle including the magnetic core 12 presenting the target analyte 20 that will remain held in the first zone by the magnet. Meanwhile, the signal component 16, to which the antibody unit 14,18 and target analyte 20 are bound, is free to migrate away from the first zone. Therefore, detection of the signal component 16 outside of the first zone will occur when the sample contains the target analyte 20.

In some embodiments, in addition to use of aptamers and antibodies, the presently-disclosed subject matter can make use of a variety of molecular recognition elements, such as, for example, the following. Short chains of amino acids (relatively shorter polypeptides, which are often referred to in the art as peptides) can be designed or selected to bind to specific targets. Peptides can be engineered to mimic the binding properties of antibodies or aptamers. Custom-designed synthetic polymers, also known as molecularly imprinted polymers (MIPs), can be created to specifically bind to certain target molecules. MIPs are polymer matrices with binding sites that are molecularly shaped to match the target molecule. Nanobodies, also known as single-domain antibodies or VHHs (variable heavy domains of heavy-chain antibodies), nanobodies are derived from the unique heavy-chain-only antibodies found in camelids. They are small, stable, and can be engineered to bind with high specificity to a wide range of target molecules. Ribozymes and deoxyribozymes, which are RNA and DNA molecules, respectively, not only bind to target molecules but also possess enzymatic activity. They can catalyze specific reactions in the presence of their target molecules, making them useful in certain applications. Chemical ligands are a type of small molecule that can be chemically designed to specifically interact with certain target molecules, making them useful for molecular recognition applications. Instead of using whole antibodies, specific binding domains of antibodies (such as Fab fragments or single-chain variable fragments, scFv) can be engineered for target binding. These antibody fragments retain the binding specificity but are smaller and can be easier to work with.

The presently-disclosed subject matter further includes a method for detecting a small molecule, a nucleotide, or a polypeptide target analyte, which involves delivering a sample to a device as disclosed herein such that the sample enters the first zone, and detecting a location of the signal component, such that (i) in the absence of the target analyte, the signal component remains bound to the magnetic core and held within the first zone, and (ii) in the presence of the target analyte, the signal component is free from the magnetic core, allowing movement away from the first zone.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see. Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about.” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The terms “nucleotide”, “polynucleotide”, “nucleic acid” and “nucleic acid sequence” refer to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides.

The term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in 5′ to 3′ direction. As is also known in the art, two sequences that hybridize to each other under a given set of conditions do not necessarily have to be 100% fully complementary. Indeed, it can be useful to design certain nucleotide sequences to be less than fully complementary, such that they have a reduced affinity for a particular sequence.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted.

The term “small molecule” as used herein, refers to organic or inorganic molecules either synthesized or found in nature, generally having a molecular weight less than 10,000 grams per mole, optionally less than 5,000 grams per mole, and optionally less than 2,000 grams per mole.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1—Detection of Target Nucleotide

The present study relates to an embodiment similar to that which is depicted in FIG. 2. For purposes of confirming utility, survivin was selected as the target nucleotide. Survivin is a representative portion of a gene that regulates cell division and inhibits apoptosis, displaying overexpression in most human cancers.¹⁻² Bladder cancer diagnosis has been linked to urine survivin while serum levels can be used for diagnosis of other types of cancer.³⁻⁴

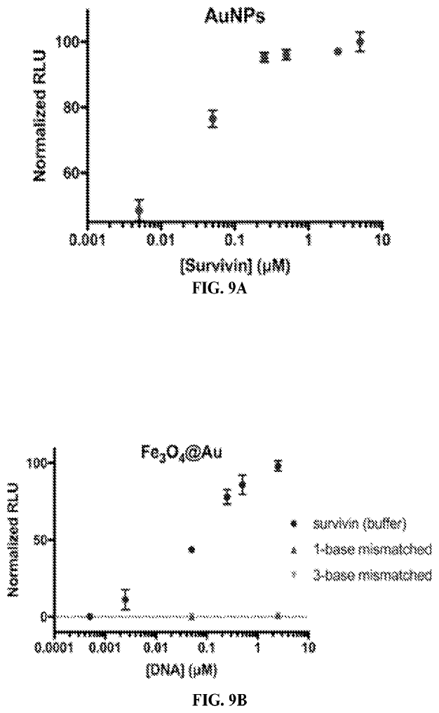

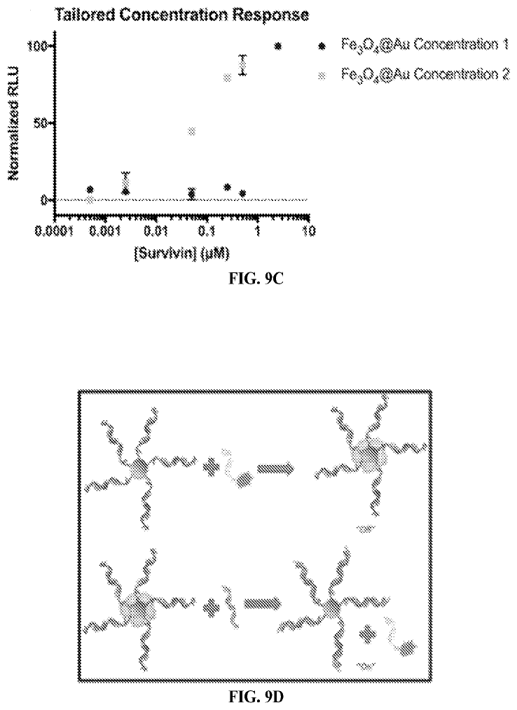

Before testing the magnetic nanoparticle within a device, the mechanism was tested in solution. As shown in FIG. 9D, the fluorescence from the fluorophore tagged flare sequence (recognition element and signal component) will largely be quenched when base-paired with the probe and in close proximity to the surface of the nanoparticles. The presence of a reduced emission from fluorophore (signal component) indicated the presence of the recognition element and signal component bound to the probe on the nanoparticle surface.

The nucleotide-decorated nanoparticles were then incubated with the target nucleotide (survivin oligonucleotides) at differing concentrations. As the recognition element and signal component is replaced by the target nucleotide, the signal component (fluorophore) will move away from the surface of the nanoparticle and the full fluorescence emission will be restored.

The data provided in FIG. 9A demonstrates that the system works on gold nanoparticles (AuNPs), with increasing fluorescence signal resulting from increasing survivin concentrations. In FIGS. 9B and 9C, AuNPs are replaced with Fe₃O₄@Au nanoparticles that can be used with a device of the presently-disclosed subject matter, which employs a magnet.

The fluorescence signal increased with increasing survivin concentrations, with detection starting in the nanomolar concentration range (and picomole amounts of DNA). In order to demonstrate selectivity, the nanoparticle formulations were also exposed to mis-matched DNA sequences, with sequences differing by one or three bases from the signal component sequence. As seen in FIG. 9B, no change in fluorescence signal was observed unless the specific target DNA sequence was present.

The data provided in FIG. 9C establishes that the signal production can be tailored to occur at different target nucleotide concentrations by altering the nanoparticle concentrations used for the analysis. With the first concentration (blue circles), the change in fluorescence was not observed until the DNA concentration moved about the 0.5 μM concentration to the 2.5 μM level. However, with second nanoparticle, the change in fluorescence signal was observed starting at 2.5 nM.

### Example 2—Detection of Target Nucleotide in Device Embodiment

Upon establishing the utility of the mechanism in solution, the utility of the magnetic nanoparticle within an exemplary embodiment of a device in accordance with the presently-disclosed subject matter. In this study, survivin analysis was demonstrated on a uPAD paper-based platform, employing a magnet, as illustrated in the schematic shown in FIGS. 1A and 1B.

μPADs were fabricated by wax-printing with a Xerox Colorqube 8570 printer on Whatman 3 MM chromatography paper. The devices were wrapped in aluminum foil and baked at (95° C.) for 4 min on the middle rack. The devices were positioned with a magnet under the first zone (analysis zone 1). Probe-labelled nanoparticles were added to either buffer or artificial saliva with differing concentrations of survivin DNA. The solutions were then added to the first zone (analysis zone 1). After the solutions migrated away from the first zone, i.e., toward analysis zone 2, the fluorescence was measured in analysis zone 2.

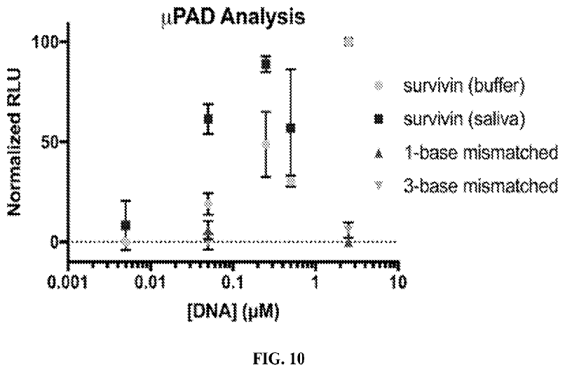

As shown in FIG. 10, the fluorescence signal increased with increasing survivin concentration. However, when exposed to the 1- and 3-base mismatched DNA no significant change in fluorescence was observed. The dip in fluorescence signal at higher concentrations may be an example of the hook effect, observed with other later flow assays.⁵

### Example 3—Detection of Target Polypeptide, Protease

The present study relates to an embodiment similar to that which is depicted in FIG. 3. For purposes of confirming utility, trypsin was selected as the target protease.

A probe polypeptide was provided with a recognition element consisting of the specific amino acid sequence corresponding to a trypsin recognition sequence, and was designed and attached to the surface of both AuNPs and Fe₃O₄@Au's. The probe/recognition element polypeptide was also labelled with a fluorophore (signal component) on the end opposite the nanoparticle conjugation.

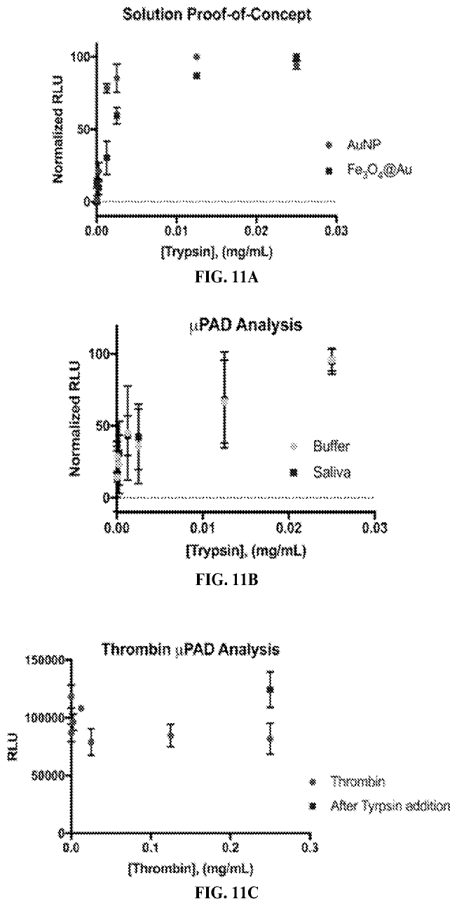

The nanoparticle systems were verified to work in solution with fluorescence quenching monitoring, as above with the exemplary nucleotide system. As shown in FIG. 11A, with both the AuNPs and Fe₃O₄@Au nanoparticle systems, the fluorescence intensity increased with increasing trypsin concentration.

With reference to FIG. 11B, in a similar fashion as the exemplary nucleotide analysis system, trypsin probe-labelled Fe₃O₄@Au nanoparticles were added to either buffer or artificial saliva with differing concentrations of trypsin. The solutions were then added to the first zone (uPAD analysis zone 1) with a magnet fixed below. After the solutions migrated away from the first zone to analysis zone 2, the fluorescence was measured in analysis zone 2.

As shown in FIG. 11C, the fluorescence signal increased with increasing trypsin concentration on the uPAD as well. However, when exposed to the thrombin, a common blood protease, no significant change in fluorescence was observed. After no significant increase in fluorescence was observed, trypsin was added to the solution and an increase in fluorescence was observed.

### Example 4—Detection of Target Small Molecule, Polypeptide, or Other Analyte Recognized by an Aptamer

The present study relates to an embodiment similar to that which is depicted in FIG. 6. For purposes of confirming utility, cortisol was selected as the target protease.

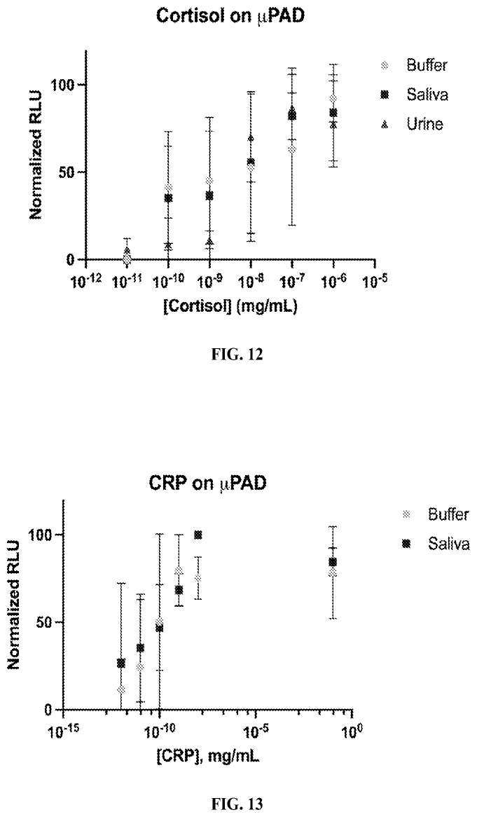

FIG. 12 includes data collected the aptamer-based detection system in buffer, urine, and saliva matricies. All points represent the mean of three replicates±one standard deviation. As shown in FIG. 12, with the aptamer-based system, the fluorescence intensity increased with increasing cortisol concentration.

### Example 5—Detection of Target Small Molecule, Polypeptide, or Other Analyte Recognized by an Antibody

The present study relates to an embodiment similar to that which is depicted in FIG. 8. For purposes of confirming utility, C-reactive protein (CRP) was selected as the target analyte.

FIG. 13b includes data collected the antibody-based detection system in buffer and saliva matricies. All points represent the mean of three replicates±one standard deviation. As shown in FIG. 13, with the antibody-based system, the fluorescence intensity increased with increasing CRP concentration.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Kennedy, S. M.; O'Driscoll, L.; Purcell, R.; Fitz-Simons, N.; McDermott, E. W.; Hill, A. D.; O'Higgins, N. J.; Parkinson, M.; Linehan, R.; Clynes, M., Prognostic importance of survivin in breast cancer. Br J Cancer 2003, 88(7), 1077-1083.
2. Jaiswal, P. K.; Goel, A.; Mittal, R. D., Survivin: A molecular biomarker in cancer. Indian J Med Res 2015, 141(4), 389-397.
3. Smith. S. D.; Wheeler. M. A.; Plescia, J.; Colberg. J. W.; Weiss, R. M.; Altieri, D. C., Urine Detection of Survivin and Diagnosis of Bladder Cancer. JAMA 2001, 285(3), 324-328.

4 Gunaldi, M.; Isiksacan, N.; Kocoglu, H.; Okuturlar, Y.; Gunaldi, O.; Topcu, T.; Karabulut, M., The value of serum survivin level in early diagnosis of cancer. Journal of Cancer Research and Therapeutics 2018, 14(3), 570-573.
5. Ross, G. M. S.; Filippini, D.; Nielen, M. W. F.; Salentijn, G. I. J., Unraveling the Hook Effect: A Comprehensive Study of High Antigen Concentration Effects in Sandwich Lateral Flow Immunoassays. Analytical Chemistry 2020, 92(23), 15587-15595.
6. Chen, Y.; Ding, X.; Zhang, Y.; Natalia, A.; Sun, X.; Wang, Z.; Shao, H., Design and synthesis of magnetic nanoparticles for biomedical diagnostics. Quantitative imaging in medicine and surgery 2018, 8(9), 957-970.
7. Bilal. M.; Zhao. Y.; Rasheed. T.; Iqbal. H. M. N., Magnetic nanoparticles as versatile carriers for enzymes immobilization: A review. International Journal of Biological Macromolecules 2018, 120, 2530-2544.

8 Lee, J. W.; Choi, S.-R.; Heo, J. H., Simultaneous Stabilization and Functionalization of Gold Nanoparticles via Biomolecule Conjugation: Progress and Perspectives. ACS Applied Materials & Interfaces 2021, 13(36), 42311-42328.
9. Eivazzadeh-Keihan, R.; Bahreinizad, H.; Amiri, Z.; Aliabadi, H. A. M.; Salimi-Bani, M.; Nakisa, A.; Davoodi. F.; Tahmasebi, B.; Ahmadpour, F.; Radinekiyan, F.; Maleki. A.; Hamblin, M. R.; Mahdavi. M.; Madanchi, H., Functionalized magnetic nanoparticles for the separation and purification of proteins and peptides. TrAC Trends in Analytical Chemistry 2021, 141, 116291.
10. Zhao, L.; Li, L.; Zhu, C.; Ghulam, M.; Qu, F., pH-responsive polymer assisted aptamer functionalized magnetic nanoparticles for specific recognition and adsorption of proteins. Analytica Chimica Acta 2020, 1097, 161-168.
11. Fresco-Cala. B.; Batista. A. D.; Cárdenas. S., Molecularly Imprinted Polymer Micro- and Nano-Particles. A review. Molecules (Basel, Switzerland) 2020, 25(20).
12. Kozlowski, R.; Ragupathi, A.; Dyer, R. B., Characterizing the Surface Coverage of Protein-Gold Nanoparticle Bioconjugates. Bioconjugate chemistry 2018, 29(8), 2691-2700.
13. Toseland, C. P., Fluorescent labeling and modification of proteins. Journal of chemical biology 2013, 6(3), 85-95.
14. Zhang, W.; Wang, R.; Luo, F.; Wang, P.; Lin, Z., Miniaturized electrochemical sensors and their point-of-care applications. Chinese Chemical Letters 2020, 31(3), 589-600.
15. Bilal, M.; Zhao, Y.; Rasheed. T.; Iqbal, H. M. N., Magnetic nanoparticles as versatile carriers for enzymes immobilization: A review. International Journal of Biological Macromolecules 2018, 120, 2530-2544.
16. Lee, J. W.; Choi, S.-R.; Heo, J. H., Simultaneous Stabilization and Functionalization of Gold Nanoparticles via Biomolecule Conjugation: Progress and Perspectives. ACS Applied Materials & Interfaces 2021, 13(36), 42311-42328.
17. Eivazzadeh-Keihan, R.; Bahreinizad, H.; Amiri, Z.; Aliabadi, H. A. M.; Salimi-Bani. M.; Nakisa. A.; Davoodi. F.; Tahmasebi, B.; Ahmadpour, F.; Radinekiyan, F.; Maleki. A.; Hamblin, M. R.; Mahdavi, M.; Madanchi, H., Functionalized magnetic nanoparticles for the separation and purification of proteins and peptides. TrAC Trends in Analytical Chemistry 2021, 141, 116291.
18. Zhao. L.; Li. L.; Zhu. C.; Ghulam. M.; Qu. F., pH-responsive polymer assisted aptamer functionalized magnetic nanoparticles for specific recognition and adsorption of proteins. Analytica Chimica Acta 2020, 1097, 161-168.
19. Fresco-Cala, B.; Batista, A. D.; Cárdenas, S., Molecularly Imprinted Polymer Micro- and Nano-Particles. A review. Molecules (Basel. Switzerland) 2020, 25(20).
20. Hurst, S. J.; Lytton-Jean, A. K.; Mirkin, C. A., Maximizing DNA loading on a range of gold nanoparticle sizes. Anal Chem 2006, 78(24), 8313-8.
21. Olsen, J. V.; Ong, S.-E.; Mann, M., Trypsin Cleaves Exclusively C-terminal to Arginine and Lysine Residues *. Molecular & Cellular Proteomics 2004, 3(6), 608-614.
22. Yu, Z.; Visse, R.; Inouye, M.; Nagase, H.; Brodsky, B., Defining requirements for collagenase cleavage in collagen type III using a bacterial collagen system. J Biol Chem 2012, 287(27), 22988-22997.
23. Coombs, G. S.; Bergstrom, R. C.; Pellequer, J.-L.; Baker, S. I.; Navre, M.; Smith, M. M.; Tainer, J. A.; Madison, E. L.; Corey, D. R., Substrate specificity of prostate-specific antigen (PSA). Chemistry & Biology 1998, 5(9), 475-488.
24. Du X, Li Y, Xia Y L, Ai S M, Liang J, Sang P, Ji X L, Liu S Q. Insights into Protein-Ligand Interactions: Mechanisms, Models, and Methods. Int J Mol Sci. 2016 Jan. 26; 17(2):144.
25. Zer C, Avery K N, Meyer K, Goodstein L, Bzymek K P, Singh G, Williams J C. Engineering a high-affinity peptide binding site into the anti-CEA mAb M5A. Protein Eng Des Sel. 2017 Jun. 1; 30(6):409-417.
26. Parker, B. W.; Goncz, E. J.; Krist, D. T.; Statsyuk, A. V.; Nesvizhskii, A. I.; Weiss, E. L. Mapping low-affinity/high-specificity peptide-protein interactions using ligand-footprinting mass spectrometry. Proceedings of the National Academy of Sciences 2019, 116 (42), 21001-21011.
27. Nguyen, H. Q.; Roy, J.; Harink, B.; Damle, N. P.; Latorraca, N. R.; Baxter, B. C.; Brower, K.; Longwell, S. A.; Kortemme, T.; Thorn, K. S.; et al. Quantitative mapping of protein-peptide affinity landscapes using spectrally encoded beads. eLife 2019, 8, e40499.
28. Kohlberger, M.; Gadermaier, G. SELEX: Critical factors and optimization strategies for successful aptamer selection. Biotechnology and Applied Biochemistry 2022, 69(5), 1771-1792. DOI: 10.1002/bab.2244.
29. Yüce, M.; Ullah, N.; Budak, H. Trends in aptamer selection methods and applications. Analyst 2015, 140(16), 5379-5399, 10.1039/C5AN00954E. DOI: 10.1039/C5AN00954E.

It will be understood that various details of the presently disclosed subject matter can be changed without departing from the scope of the subject matter disclosed herein. Furthermore, the foregoing description is for the purpose of illustration only, and not for the purpose of limitation.

1. A device for detecting a target analyte in a sample, comprising:
a) a region, comprising
i) a first zone, into which the sample can be delivered; and
ii) a second zone in fluid communication with the first zone;
b) a magnet positioned adjacent the first zone; and
c) a magnetic nanoparticle held within the first zone by the magnet, comprising a magnetic core.

2. The device according to claim 1, and further comprising:
a signal component;
a recognition element that is covalently or non-covalently conjugated to the signal component; and
a probe that (i) is covalently or non-covalently conjugated to the recognition element, or (ii) taken together with the recognition element, forms a single unit;
wherein the probe, the recognition element, or the signal component has an affinity for the target analyte, or wherein the target analyte cleaves the recognition element, such that in the presence of the target analyte, the signal component is free from the magnetic core.

3. The device of claim 2, wherein the probe is covalently or non-covalently conjugated to the magnetic core.

4. The device of claim 3, wherein the recognition element has an affinity for the probe, and a stronger affinity for the target analyte, such that
in the absence of the target analyte, the signal component is bound to the magnetic core due to the affinity between the probe and the recognition element, and
in the presence of the target analyte, the signal component is free from the magnetic core due to the stronger affinity between the recognition element and the target analyte.

5. The device of claim 4, wherein
the target analyte is a target nucleotide;
the probe is a probe nucleotide; and
the recognition element is a recognition nucleotide conjugated to the probe nucleotide through complementary base-pairing;
wherein the recognition nucleotide has an affinity for the probe nucleotide, and a stronger affinity for the target nucleotide.

6. The device of claim 3, wherein the probe has an affinity for the recognition element, and a stronger affinity for the target analyte, such that
in the absence of the target analyte, the signal component is bound to the magnetic core due to the affinity between the probe and the recognition element, and
in the presence of the target analyte, the signal component is free from the magnetic core due to the stronger affinity between the probe and the target analyte.

7. The device of claim 6, wherein
the target analyte is a target nucleotide;
the probe is a probe nucleotide; and
the recognition element is a recognition nucleotide conjugated to the probe nucleotide through complementary base-pairing;
wherein the probe nucleotide has an affinity for the recognition nucleotide, and a stronger affinity for the target nucleotide.

8. The device of claim 3, wherein
the target analyte is a protease;
the probe and the recognition element, taken together, form a single unit that is a polypeptide; and
the recognition element is an amino acid sequence recognized by the protease for cleaving, such that,
in the absence of the target protease, the signal component is bound to the magnetic core due to polypeptide remaining intact, and
in the presence of the target protease, polypeptide is cleaved, such that the signal component is free from the magnetic core.

9. The device of claim 3, wherein
the target analyte is a polypeptide or small molecule;
the recognition element has an affinity for the signal component, and a stronger affinity for the target analyte, such that,
in the absence of the target analyte, the signal component is bound to the magnetic core due to the affinity between the recognition element and the signal component; and
in the presence of the target polypeptide, the signal component is free from the magnetic core due to the stronger affinity between the recognition element and the target polypeptide.

10. The device of claim 3, wherein
the target analyte is a polypeptide or small molecule;
the signal component has an affinity for the recognition element, and a stronger affinity for the target analyte, such that,
in the absence of the target analyte, the signal component is bound to the magnetic core due to the affinity between the recognition element and the signal component; and
in the presence of the target polypeptide, the signal component is free from the magnetic core due to the stronger affinity between the signal component and the target polypeptide.

11. The device of claim 3, wherein
the target analyte can be recognized by an aptamer; and
the recognition element is an aptamer, and the probe and the recognition element, taken together, form a single unit non-covalently attached to the magnetic core;
wherein the aptamer has a stronger affinity for the target analyte than for the magnetic core, such that
in the absence of the target analyte, the signal component is bound to the magnetic core due to the affinity between the aptamer and the magnetic core, and
in the presence of the target analyte, the signal component is free from the magnetic core due to the stronger affinity between the aptamer and the target analyte.

12. The device of claim 4, wherein
the target analyte can be recognized by an aptamer;
the probe is a probe nucleotide; and
the recognition element is a nucleotide aptamer conjugated to the probe nucleotide through complementary base-pairing;
wherein the nucleotide aptamer has an affinity for the probe nucleotide, and a stronger affinity for the target analyte.

13. The device of claim 1, and further comprising:
target analytes covalently or non-covalently bound to the magnetic core; and
a prepared sample, including a recognition element and a probe, taken together to form a single unit attached to a signal component, such that
in the absence of the target analyte in the sample, the signal component is bound to the magnetic core due to the affinity between the single unit and the target analytes bound to the magnetic core, and
in the presence of the target analyte in the sample, the signal component is free to migrate away from the magnetic core because the single unit was bound to the target analyte in the sample, such that it is unable to bind the target analytes bound to the magnetic core.

14. The device of claim 13, wherein the target analyte can be recognized by an antibody or fragment thereof, and the single unit includes an antibody or fragment thereof that selectively binds the target analyte.

15. The device of claim 13, wherein the target analyte can be recognized by an aptamer, and the single unit includes an aptamer that selectively binds the target analyte.

16. The device of claim 13, wherein the target analyte can be recognized by a nucleotide, and the single unit includes a nucleotide that selectively binds the target analyte.

17. The device of claim 13, wherein the target analyte can be recognized by a polypeptide, and the single unit includes a polypeptide that selectively binds the target analyte.

18. A method for detecting a target analyte, comprising:
a) delivering a sample to the device of claim 1, such that the sample enters the first zone;
b) detecting a location of the signal component, such that
in the absence of the target analyte, the signal component is bound to the magnetic core and held within the first zone, and
in the presence of the target analyte, the signal component is free from the magnetic core, allowing movement away from the first zone.

19. A kit for use in detecting a target analyte in a sample, comprising:
a) a device including
i) a region, comprising
a first zone, into which the sample can be delivered; and
a second zone in fluid communication with the first zone;
ii) a magnet positioned adjacent the first zone;
iii) a magnetic nanoparticle held within the first zone by the magnet, comprising a magnetic core;
b) a probe; and
c) a recognition element that can be covalently or non-covalently conjugated to a signal component.

20. The kit of claim 19, and further comprising a signal component.

21-25. (canceled)
